(19) United States
(12) Patent Application Publication
(10) Pub. No.: US 2024/0115597 A1
(43) Pub. Date: Apr. 11, 2024

(54) METHOD FOR TREATING AND/OR PREVENTING HEARING LOSS BY USING TOLL-LIKE RECEPTOR 7 AND/OR 9 ANTAGONIST

(71) Applicant: Kaohsiung Chang Gung Memorial Hospital, Kaohsiung City (TW)

(72) Inventor: Chao-Hui Yang, Kaohsiung City (TW)

(21) Appl. No.: 18/229,018

(22) Filed: Aug. 1, 2023

(30) Foreign Application Priority Data
Oct. 6, 2022 (TW) 111138092

(51) Int. Cl. A61K 31/711; A61K 31/4706; A61P 27/16

(52) U.S. Cl. CPC A61K 31/711; A61K 31/4706; A61P 27/16

(57) ABSTRACT

The present disclosure provides a method for treating and/or preventing hearing loss by using a Toll-like receptor 7 and/or 9 antagonist. The Toll-like receptor 7 and/or 9 antagonist of the present disclosure achieves the effect of treating and/or preventing hearing loss by attenuating noise-induced increased gene expression of cytokines and chemokines in the cochlea, attenuating the noise-induced increase of nuclear factor kappa-B expression in the cochlea, and decreasing noise-induced macrophage infiltration into the cochlea.

### CROSS-REFERENCE TO RELATED APPLICATION

This application claims priority of Taiwan patent application No. 111138092, filed on Oct. 6, 2022, the content of which is incorporated herein in its entirety by reference.

### BACKGROUND OF THE INVENTION

### 1. Field of the Invention

The present invention relates to a method for treating and/or preventing hearing loss by using a Toll-like receptor 7 and/or 9 antagonist (TLR7/9 antagonist).

### 2. The Prior Art

In modern society, hearing loss has become one of the great concern diseases and around 466 million people worldwide have disabling hearing loss. Most hearing loss is sensorineural, and there is no good regimen in the clinic to recover the hearing once the hair cells die after injury. Particularly, acute sensorineural hearing loss is one of the main patterns of hearing loss in modern society. It includes noise-induced hearing loss caused by acoustic trauma due to industrial, military or recreational noise, and idiopathic sudden sensorineural hearing loss, in which the etiology is unknown.

In acute sensorineural hearing loss, the formation of reactive oxygen species (ROS) in outer hair cells (OHCs) was thought as an essential mediator for cell death. However, the involvement of immune-inflammatory response was hypothesized in the mechanism of acute sensorineural hearing loss recently. For example, Toll-like receptor (TLR4) immunostaining intensity was significantly increased in the cochlea of rats after acoustic trauma. We also observed increased expression of TLR2 in the peripheral blood leukocytes of the patients with idiopathic sudden sensorineural hearing loss and associated with hearing severity. However, it is unknown whether the usage of TLR inhibitors could protect or treat acute sensorineural hearing loss or not.

At present, the traditional treatment of acute sensorineural hearing loss mostly uses steroid drugs, but the side effects of steroids are serious. In order to solve the above-mentioned problems, those skilled in the art urgently need to develop a novel medicament for treating and/or preventing hearing loss with less side effects for the benefit of a large group of people in need thereof.

### SUMMARY OF THE INVENTION

A primary objective of the present invention is to provide a method for treating and/or preventing hearing loss, comprising administering to a subject in need thereof a pharmaceutical composition comprising an effective amount of a Toll-like receptor 7 and/or 9 antagonist (TLR7/9 antagonist).

According to an embodiment of the present invention, the hearing loss is acute sensorineural hearing loss.

According to an embodiment of the present invention, the acute sensorineural hearing loss includes noise-induced hearing loss and idiopathic sudden sensorineural hearing loss.

According to an embodiment of the present invention, the structural change of noise-induced hearing loss is noise-induced outer hair cell loss.

According to an embodiment of the present invention, the TLR7/9 antagonist attenuates noise-induced increased gene expression of cytokines and chemokines in the cochlea.

According to an embodiment of the present invention, the TLR7/9 antagonist attenuates noise-induced increase of nuclear factor kappa-B (NF-κB) expression in the cochlea.

According to an embodiment of the present invention, the TLR7/9 antagonist decreases noise-induced macrophage infiltration into the cochlea.

According to an embodiment of the present invention, the TLR7/9 antagonist is a quinine drug or oligodeoxynucleotide 2088 (ODN 2088).

According to an embodiment of the present invention, the quinine drug is chloroquine or hydroxychloroquine.

According to an embodiment of the present invention, the effective amount of the chloroquine is 40-60 mg/kg of the subject.

According to an embodiment of the present invention, the effective amount of the ODN 2088 is 4-6 mg/kg of the subject.

In summary, the TLR7/9 antagonist of the present invention has the effects on attenuating noise-induced increased gene expression of cytokines and chemokines in the cochlea, attenuating the noise-induced increase of NF-κB expression in the cochlea, and decreasing noise-induced macrophage infiltration into the cochlea, thereby achieving the effect on treating and/or preventing hearing loss.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included here to further demonstrate some aspects of the present invention, which can be better understood by reference to one or more of these drawings, in combination with the detailed description of the embodiments presented herein.

FIGS. 1A and 1B show that chloroquine and ODN 2088 attenuate 106-dB noise-induced auditory threshold shifts 3 weeks after 106-dB exposure. Number=6 in each group, *, P<0.05, **, P<0.01.

FIGS. 2A and 2B show that chloroquine and ODN 2088 attenuate 106-dB noise-induced wave I amplitude reduction at 16 kHz. Number=6 in each group, *, P<0.05.

FIG. 3 shows that chloroquine decreases noise-induced outer hair cell (OHC) loss. Number=3-4 in each group.

FIGS. 4A and 4B show that systemic (intraperitoneal) chloroquine administration decreases noise-induced elevation of cochlear cytokines and chemokines expression, in which in FIG. 4A, Tlr9 represents Toll-like receptor 9, MyD88 represents Myeloid differentiation primary response 88, Il-1β represents interleukin-1β, Tnf-α represents tumor necrosis factor-α, and Irf-7 represents Interferon regulatory factor 7; in FIG. 4B, Ccl2 represents C-C motif chemokine ligand 2, Ccl4 represents C-C motif chemokine ligand 4, and Ccl12 represents C-C motif chemokine ligand 12. Number=5-7 in each group.

FIG. 5 shows that chloroquine attenuates noise-induced nuclear factor kappa-B (NF-κB) immunostaining of the cochlea. Number=3-4 in each group.

FIGS. 6A and 6B show that chloroquine attenuates noise-induced infiltration of Iba-1±macrophages (red arrowheads in FIG. 6A and white arrows in FIG. 6B) in the lower spiral ligament. Quantification reveals chloroquine reduces the number of Iba-1±macrophage infiltration in the cochlea. Number=3-4. **, P<0.01. ***, P<0.001.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, the pharmaceutical composition provided according to the present invention can be in any suitable form, without any special limitation, depending on the intended use and the corresponding suitable dosage form. By way of example, but not limitation, the pharmaceutical composition may be administered to a subject in need thereof by oral or parenteral (e.g., transdermal or intratympanic injection) administration.

The pharmaceutical composition according to the present invention may comprise a pharmaceutically acceptable carrier widely used in pharmaceutical manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

Taking the dosage form suitable for transdermal administration as an example, the pharmaceutical composition provided according to the present invention can be in the form of patches, lotions, creams, gels (for example: hydrogels), pastes (for example: dispersion paste, ointment), spray, or solution (for example: suspension), etc., but not limited to this.

According to the present invention, the pharmaceutical composition can be manufactured to an external preparation suitable for topical application to the skin using techniques well known to those skilled in the art, including, but not limited to, emulsion, gel, ointment, cream, patch, liniment, powders, aerosol, spray, lotion, serum, paste, foam, drop, suspension, salve, and bandage.

According to the present invention, the external preparation is prepared by mixing the pharmaceutical composition of the present invention with a base well known to those skilled in the art.

According to the present invention, the base may comprise one or more additives selected from the group consisting of water, alcohols, glycol, hydrocarbons such as petroleum jelly and white petrolatum, wax such as paraffin and yellow wax, preserving agents, antioxidants, surfactants, absorption enhancers, stabilizing agents, gelling agents such as carbopol® 974P, microcrystalline cellulose and carboxymethylcellulose, active agents, humectants, odor absorbers, fragrances, pH adjusting agents, chelating agents, emulsifiers, occlusive agents, emollients, thickeners, solubilizing agents, penetration enhancers, anti-irritants, colorants, and propellants. The selection and quantity of these additives fall within the scope of professional literacy and routine techniques of those skilled in the art.

The pharmaceutical composition provided according to the present invention can be administered with different administration frequencies such as once a day, multiple times a day, or once a few days, depending on the individual's needs, age, weight, and health status. In the pharmaceutical composition provided according to the present invention, the content ratio of Toll-like receptor 7 and/or 9 antagonist (TLR7/9 antagonist) in the pharmaceutical composition can be adjusted according to actual application requirements. In addition, the pharmaceutical composition may additionally contain one or more other active ingredients (e.g., medications for hearing loss) as needed, or be used in combination with a drug containing the one or more other active ingredients to further enhance the efficacy of the drug or increase the flexibility and compounding degree of the formulation, as long as the other active ingredients are effective against the active ingredients of the present invention (that is, TLR7/9 antagonist) without adverse effects.

According to the present invention, researchers are increasingly focusing on the use of TLR7/9 antagonists. Antagonists include chloroquine, hydroxychloroquine, inhibitory oligodeoxynucleotide (ODN) (i.e., ODN2088), or novel small molecule TLR7/9 antagonists. The present invention provides a TLR7/9 antagonist that can protect or treat hearing loss, such as acute sensorineural hearing loss, including noise-induced hearing loss and idiopathic sudden sensorineural hearing loss.

According to the present invention, the types of TLR7/9 antagonists can be referred to those described in Siquan Sun et al., (2007), Inflammation & Allergy-Drug Targets, 6, 223-235; Marc Lamphier et al., (2013), Molecular Pharmacology, DOI: 10.1124/mol.113.089821; and Christopher P. et al., (2020), ACS Med. Chem. Lett., 11, 1751-1758. The present invention is not limited.

### Example 1

### TLR7/9 Antagonists Attenuate Noise-Induced Auditory Brainstem Response (ABR) Threshold Shift

Two TLR7/9 inhibitors were tested to see their protective effects on noise-induced hearing loss. The first inhibitor was chloroquine (Sigma-Aldrich, St. Louis, MO, USA), which could mask the TLR7/9 ligand binding site. The second inhibitor was suppressive ODN (ODN 2088)(Invivogen, San Diego, CA, USA), which is a short single-stranded oligodeoxynucleotide (ODN) that can inhibit the activation of TLR7 and TLR9.

The animal models used in this example and the following examples are as follows. Male CBA/CaJ mice at 4-6 weeks of age were purchased from National Laboratory Animal Center in Taiwan. The mice accessed to water and a regular mouse diet, and were kept at 22±1° C. under a standard 12h:12h light-dark cycle to acclimate for one week before the experiments. The lights were on from 5:00 to 17:00).

The experimental procedure of noise exposure is as follows. Three awake CBA/CaJ mice in the same group were placed in a sound chamber, with one mouse per stainless steel cage. The sound chamber was fitted with a loudspeaker, driven by an amplifier connected to a CD player. Broadband noise (BBN) with a frequency of 2 to 20 kHz was compiled onto a CD and equalized with software. Sound levels were calibrated with a sound level meter (type 2250; Brtiel & Kja✓r) fitted with a microphone (type 4189; Brtiel & Kja✓r, Na✓rum, Denmark) at the locations of each cage within the sound chamber, before and after exposure to the noise. The control (sham) group was put in the same cage without exposure to noise for 2 hours. An intensity of 106 dB broadband noise was used to produce permanent threshold shifts.

The experimental procedure of TLR7/9 antagonists treatment is as follows. Chloroquine was dissolved in saline. ODN 2088 was dissolved in endotoxin-free water. The control group was endotoxin-free water. The route of chloroquine and ODN 2088 administration was via intraperitoneal and subcutaneous routes, respectively. The dosage of chloroquine was set as 50 mg/kg of body weight. ODN 2088 was set as 5 mg/kg of body weight. These dosages are shown based on our testing that did not cause ototoxicity. For the mice used for experiments to investigate the expression of TLR7/9 signaling molecules, three times treatments (24 hours before, 2 hours before, and immediately after noise exposure) were performed. For the mice used of experiments to observe the ABR threshold, four times treatments (24 hours before, 2 hours before, immediately and 24 hours after noise exposure) were performed.

The experimental procedure of auditory brainstem response (ABR) is as follows. Mice were anesthetized with an intraperitoneal injection of Zoletil (25 mg/kg) and Xylazine (23 mg/kg), and then placed in a sound-isolated and electrically shielded booth. Body temperature was monitored and maintained near 37° C. with a heating pad. Acoustic stimuli was delivered monaurally to an earphone inserted into the ear canal. Sub-dermal electrodes were inserted at the vertex of the skull, under the left ear and under the right ear (ground). ABRs were measured at 8, 16, and 32 kHz. Up to 1024 responses were averaged for each stimulus level. Thresholds were determined for each frequency by reducing the intensity in 10 dB increments and then in 5 dB steps near the threshold until no organized responses were detected. Thresholds were estimated between the lowest stimulus level where a response is observed and the level without response. ABR thresholds were detected at before noise exposure and one week/three week after noise exposure. The ABR threshold shift was defined as the difference between the post- and pre-noise ABR thresholds.

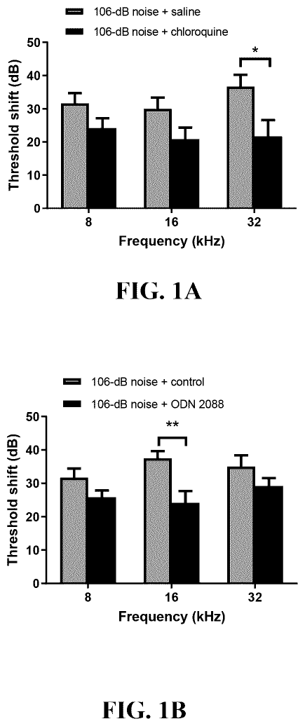

To test if TLR7/9 antagonists could protect the mice against noise-induced hearing loss, we checked the ABR threshold shift at 3 weeks after two kinds of TLR7/9 antagonists (chloroquine or ODN 2088) treatment. As shown in FIGS. 1A and 1B, 106-dB noise caused a threshold shift about 30-40 dB 3 weeks after 106-dB noise exposure, which was proved as permanent threshold shift. Chloroquine or ODN2088 treatment decreased the noise-induced ABR thresholds shift. These results revealed that TLR7/9 antagonists could attenuate noise-induced hearing loss.

### Example 2

### TLR7/9 Antagonists Attenuate Noise-Induced Reduction of ABR Wave I Amplitude

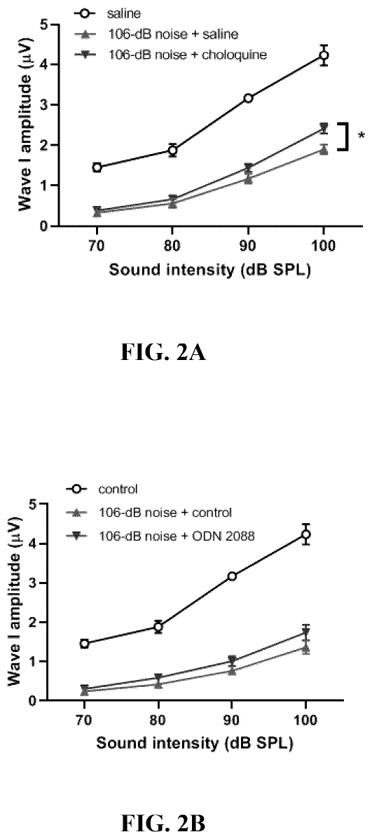

To test if TLR7/9 antagonists could protect the auditory nerve against acoustic trauma, we measured the ABR wave I amplitudes at 16 kHz, which is one of the most sensitive frequencies of the auditory spectrum in the mice. As shown in FIGS. 2A and 2B, 106-dB noise caused the decline of wave I amplitudes, whereas chloroquine (see FIG. 2A) and ODN 2088 (see FIG. 2B) attenuated the noise-induced decrease in the amplitudes. This result implies that TLR7/9 antagonists could protect against noise-induced cochlear synaptopathy.

### Example 3

### TLR7/9 Antagonist Reduces Noise-Induced Outer Hair Cell (OHC) Loss

To examine if TLR7/9 antagonist could protect the OHC from acoustic trauma, we counted the OHCs in surface preparations after 106-dB noise exposure.

The experimental procedure of surface preparation is as follows. The temporal bones were removed immediately following euthanasia and perfused through the scala with a solution of 4% paraformaldehyde in phosphate-buffered saline, pH 7.4 (PBS), and kept in this fixative overnight at 4° C. The cochleae will then be rinsed in PBS. Before decalcification of each cochlea in a 4% solution of sodium EDTA (adjusted with HCl to pH 7.4), the apical otic capsule was removed. The EDTA solution was changed daily for 3 days at 4° C. Then the cochleae were further dissected under a microscope by removing the softened optic capsule, stria vascularis, Reissner's membrane, and tectorial membrane.

The experimental procedure of immunocytochemistry for outer hair cell (OHC) counts is as follows. Upon incubating in 3% Triton X-100 in PBS for 30 min and washing 3 times with PBS, the specimens were incubated in rhodamine-phalloidin (1:100; Invitrogen, Carlsbad, CA, USA) for 60 min and followed by PBS rinses. The sections were mounted on glass slides with PermaFluorTM Mountan (Thermo Fisher Scientific, Pittsburgh, PA, USA). Cell populations in the phalloidin-stained stereociliary bundles and circumferential F-actin rings around the cuticular plate of the OHCs were evaluated using an epifluorescence microscope. The right objective had a 0.25 mm calibrated scale imposed on the field for reference, and all three rows of OHCs were oriented longitudinally within each 0.25 mm frame. Each successive 0.25 mm field was evaluated beginning from the apex to the base for counting the number of OHCs. Cell counts were entered into a computer program (KHRI Cytocochleogram, version 3.0.7) to calculate the percentage of OHC loss from the apical turn to the basal turn of the basal epithelium.

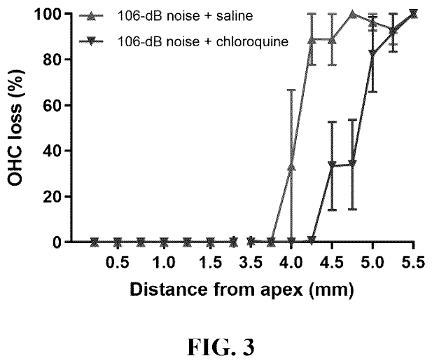

As shown in FIG. 3, noise caused the OHC loss in the basal segment, whereas chloroquine attenuated the percentage of OHC loss over this area. This result reveals the protective effect of TLR7/9 antagonist against noise-induced OHC damage.

### Example 4

### TLR7/9 Antagonist Attenuated Noise-Induced Increased Gene Expression of Cytokines and Chemokines in the Cochlea

To test if TLR7/9 antagonist could affect the expression of cochlear cytokines and chemokines after noise, we used quantitative real-time reverse transcription polymerase chain reaction (quantitative real-time RT-PCR) to analyze gene expression in the cochlea one day after systemic chloroquine treatment.

The experimental procedure of RNA isolation and quantitative real-time RT-PCR is as follows. Cochlear tissue was washed twice with PBS after incubation and kept in TRIzol until analysis. All tissues contributing to a pooled sample will undergo the same treatment conditions. Total RNA (1 lag) is reverse transcribed to first-strand cDNA by High Capacity cDNA Reverse Transcription Kit (Applied Biosystems, Foster City, CA, USA) according to the manufacturer's protocols. The cDNA was diluted (1:10) with DEPC-H2O and stored in aliquots at −20° C. Real-time quantitative PCR was performed in an ABI 7500 Fast Real-Time System (Applied Biosystems). The expression levels of the tested genes were normalized to the internal control Actb to obtain the relative threshold cycle (ACT), and the relative expression between experimental and control groups was calculated by the comparative CT (AACT) method.

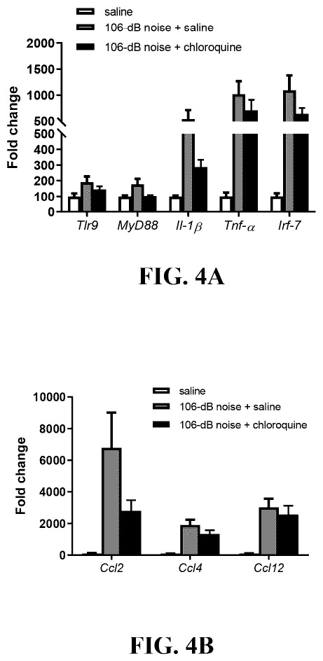

As shown in FIGS. 4A and 4B, noise decreased the mRNA expression of Toll-like receptor 9 (Tlr9), Myeloid differentiation primary response 88 (MyD88), interleukin-lfl tumor necrosis factor-α (Tnf-α) and Interferon regulatory factor 7 (Irf-7) in the cochlea, whereas chloroquine attenuated these changes. Also, chloroquine decreased the noise-induced elevation of C-C motif chemokine ligand 2 (Ccl2), C-C motif chemokine ligand 4 (Ccl4) and C-C motif chemokine ligand 12 (Ccl12) expression. These results reveal that TLR7/9 antagonists could attenuate cochlear cytokines and chemokines during acoustic trauma.

### Example 5

### TLR7/9 Antagonist Treatment Attenuates the Noise-Induced Increase of Nuclear Factor Kappa-B (NF-κB) Expression in the Cochlea

To test whether TLR7/9 antagonist could attenuate the NF-κB in the cochlea, we immunostained the middle-basal segment of the cochlea with NF-κB antibody.

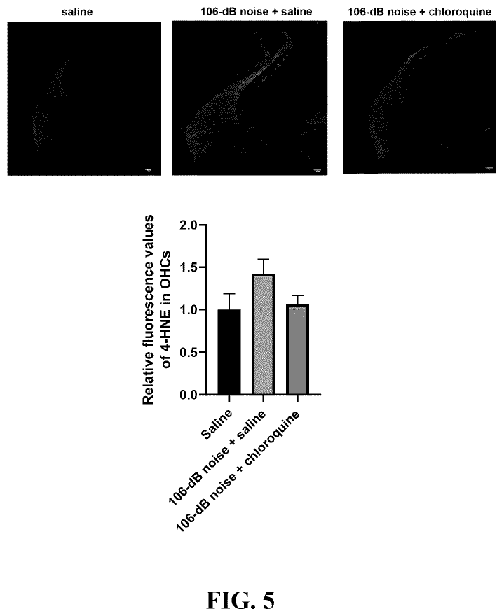

As shown in FIG. 5, immunofluorescence staining increased in the lateral wall of cochlea after 106-dB noise, while chloroquine decreased the immunostaining of NF-κB. This data implied that chloroquine could attenuate noise-induced cochlear NF-κB activation.

### Example 6

TLR7/9 Antagonist Treatment Attenuates Noise-Induced Increase of Iba-1±Cells Infiltration into the Cochlea

To test if TLR7/9 antagonist could affect macrophage infiltration after acoustic trauma, we checked the number of Iba-1±cells, which represent activated macrophages, in the lower spiral ligament of basal cochlear section.

The experimental procedure of immunohistochemistry of cochlear paraffin sections is as follows. Each cochlea was transferred to 70% ethanol after 4% EDTA and embedded in paraffin for sectioning, 5 μm sections were deparaffined in xylene and rehydrated in alcohol. The sections were incubated with target retrieval solution in a steamer for 10 min and then 3% hydrogen peroxide for 10 min and protein block for 20 min at room temperature. Primary antibodies for biomarkers were incubated overnight at 4° C., followed by biotinylated secondary antibody for 30 min and avidin-biotin complex (ABC) reagent for 30 mins Immunocomplexes of horseradish peroxidase were visualized by 3,3′-diaminobenzidine (DAB) reactions, and sections were counterstained with hematoxylin before mounting.

The experimental procedure of immunofluorescence for cryosection and surface preparation is as follows. Cochlea was permeabilized in 3% Triton X-100 solution for 30 min at room temperature. The specimens were washed three times with PBS and blocked with 10% normal goat serum for 30 min at room temperature, followed by incubation with primary antibody at 4° C. for 72 hours. After washing three times with PBS, the secondary antibody was applied at 4° C. overnight in darkness. For fluorescent visualization of hair cell structure, the specimens were incubated with appropriate phalloidin (1:100) at room temperature for 30 min. In cochlear surface preparations, the epithelia was divided into three segments (apex, middle, and base). Specimens were mounted on slides with anti-fade mounting media and imaged on a confocal microscope. In cryosected cochlea, the interested region was viewed specifically.

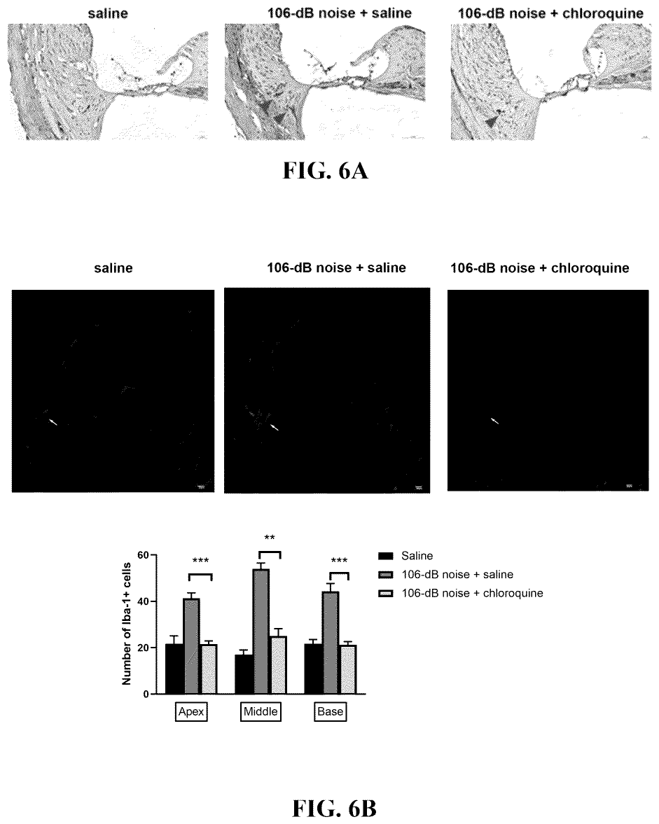

As shown in immunohistochemistry (see FIG. 6A), noise increases the number of Iba-1±cells over the lower spiral ligament of the cochlea, while chloroquine attenuates the increase in Iba-1±cell infiltration. Further quantification of the immunofluorescence for Iba-1±cells also proved that chloroquine decreases the number of Iba-1±signals in each segment of the cochlea (see FIG. 6B). This implies that chloroquine decreases noise-induced macrophage infiltration into the cochlea.

In summary, the TLR7/9 antagonist of the present invention has the effects on attenuating noise-induced increased gene expression of cytokines and chemokines in the cochlea, attenuating the noise-induced increase of NF-κB expression in the cochlea, and decreasing noise-induced macrophage infiltration into the cochlea, thereby achieving the effect on treating and/or preventing hearing loss.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating and/or preventing hearing loss, comprising administering to a subject in need thereof a pharmaceutical composition comprising an effective amount of a Toll-like receptor 7 and/or 9 antagonist (TLR7/9 antagonist).

2. The method according to claim 1, wherein the hearing loss is acute sensorineural hearing loss.

3. The method according to claim 2, wherein the acute sensorineural hearing loss includes noise-induced hearing loss and idiopathic sudden sensorineural hearing loss.

4. The method according to claim 3, wherein the noise-induced hearing loss is noise-induced outer hair cell loss.

5. The method according to claim 1, wherein the TLR7/9 antagonist attenuates noise-induced increased gene expression of cytokines and chemokines in cochlea.

6. The method according to claim 1, wherein the TLR7/9 antagonist attenuates noise-induced increase of nuclear factor kappa-B (NF-κB) expression in cochlea.

7. The method according to claim 1, wherein the TLR7/9 antagonist decreases noise-induced macrophage infiltration into cochlea.

8. The method according to claim 1, wherein the TLR7/9 antagonist is a quinine drug or oligodeoxynucleotide 2088 (ODN 2088).

9. The method according to claim 8, wherein the quinine drug is chloroquine or hydroxychloroquine.

10. The method according to claim 9, wherein the effective amount of the chloroquine is 40-60 mg/kg of the subject.

11. The method according to claim 8, wherein the effective amount of the ODN 2088 is 4-6 mg/kg of the subject.
